(19) United States
(12) Patent Application Publication
(10) Pub. No.: US 2026/0219182 A1
(43) Pub. Date: Jul. 30, 2026

(54) WATER DISPENSING APPARATUS

(71) Applicant: LG ELECTRONICS INC., Seoul (KR)

(72) Inventors: Ilna SON, Seoul (KR); Chulwoo PARK, Seoul (KR); Yumin KIM, Soul (KR); Youngjin KIM, Seoul (KR)

(21) Appl. No.: 19/148,486

(22) PCT Filed: Jun. 29, 2023
(86) PCT No.: PCT/KR2023/009116

§ 371 Date: Jul. 16, 2025

(30) Foreign Application Priority Data
Jan. 18, 2023 (KR) 10-2023-0007295

(51) Int. Cl. G01N 21/53; B67D 1/08; B67D 1/12; G01N 21/59; G01N 33/18

(52) U.S. Cl. CPC G01N 21/532; B67D 1/0857; B67D 1/0895; B67D 1/1277; G01N 21/59; G01N 33/1826; B67D 2210/0001

(57) ABSTRACT

A water dispensing apparatus according to an embodiment of the present disclosure includes: a water supply passage through which raw water, supplied from a water source, flows; a filter configured to generate purified water by filtering the raw water supplied through the water supply passage; a purified water passage through which the purified water, having passed through the filter, flows; and a water quality measurement unit connected to the purified water passage and configured to measure water quality of the purified water, wherein the water quality measurement unit includes a turbidity sensor module configured to measure turbidity of water introduced thereinto, wherein the turbidity sensor module includes: a water inlet portion for receiving water; an internal channel through which water, introduced through the water inlet portion, flows; a chamber filled with water discharged from the internal channel; a light source unit including a light source configured to emit light into the chamber; and a light receiver configured to receive scattered light that is scattered from the chamber, wherein a light path of light emitted by the light source unit is spaced apart from the internal channel.

### TECHNICAL FIELD

The present description relates to a water dispensing apparatus and a method of operating the same, and more particularly to a water dispensing apparatus including a sensor capable of determining water quality, and a method of operating the same.

### BACKGROUND ART

A water dispensing apparatus is an apparatus for supplying water and dispensing a desired amount of water at a desired temperature according to a user's operation. The water dispensing apparatus may be applied to various fields, but may be typically applied to refrigerators and water purifiers. Particularly, the water dispensing apparatus provided in the refrigerator and the water purifier may be configured to automatically supply a set amount of water according to a user's operation. Recently, water dispensing apparatuses capable of supplying not only purified water but also cold water and hot water have been developed.

For example, a water purifier is connected to a water source, such as a water faucet and the like, to receive raw water, and is configured to remove floating substances and harmful substances contained in the raw water by using a filter and to purify and dispense a desired amount of water according to a user's operation. Various purifiers are on the market, including a purifier capable of not only purifying water, but also heating or cooling purified water to supply hot water or cold water. Further, purifiers having a compact size and capable of being installed in various installation environments are being developed these days.

If the water dispensing apparatus is used for a long time, microorganisms and the like may proliferate in pipes, valves, and water outlets, or the apparatus may be contaminated, and if a filter replacement period has elapsed, floating substances and harmful substances contained in raw water may not be removed. Accordingly, it is important to measure water quality accurately and to manage the water dispensing apparatus hygienically, and purified water quality performance should be managed as well.

Korean Laid-open Patent Publication No. 10-2013-0119357 as related art discloses a turbidity sensor configured to determine turbidity of a solution based on a ratio of visible light to infrared light, but it provides no means for preventing or removing noise generated due to internal passage environment.

### DISCLOSURE OF INVENTION

### Technical Problem

It is an objective of the present disclosure to provide a water dispensing apparatus capable of measuring water quality more accurately.

It is another objective of the present disclosure to provide a water dispensing apparatus capable of effectively separating and discharging bubbles.

It is yet another objective of the present disclosure to provide a water dispensing apparatus capable of accurately measuring water quality of raw water and purified water.

It is yet another objective of the present disclosure to provide a water dispensing apparatus capable of automatically detecting abnormal water quality and managing a passage hygienically.

It is yet another objective of the present disclosure to provide a water dispensing apparatus capable of improving sensing accuracy and efficiency by a rinse operation and water pipe configuration for sharing a sensor.

### Solution to Problem

In accordance with an aspect of the present disclosure, the above and other objectives can be accomplished by providing a water dispensing apparatus including: a water supply passage through which raw water, supplied from a water source, flows; a filter configured to generate purified water by filtering the raw water supplied through the water supply passage; a purified water passage through which the purified water, having passed through the filter, flows; and a water quality measurement unit connected to the purified water passage and configured to measure water quality of the purified water, wherein the water quality measurement unit includes a turbidity sensor module configured to measure turbidity of water introduced thereinto, wherein the turbidity sensor module includes: a water inlet portion for receiving water; an internal channel through which water, introduced through the water inlet portion, flows; a chamber filled with water discharged from the internal channel; a light source unit including a light source configured to emit light into the chamber; and a light receiver configured to receive scattered light that is scattered from the chamber, wherein a light path of light emitted by the light source unit is spaced apart from the internal channel.

The turbidity sensor module may include a first water outlet portion through which water is discharged from the internal channel to the chamber, and a second water outlet portion through which water is discharged from the chamber to an outside.

The first water outlet portion may be formed in an open portion of one surface of the internal channel that faces the second water outlet portion.

One surface of the internal channel in which the first water outlet portion is formed may have an inclined portion inclined at both sides of the first water outlet portion.

A size of the second water outlet portion may be smaller than a half of a length of the internal channel.

The turbidity sensor module may further include a flow generator formed in an open portion of a side surface of the internal channel that faces the light path.

The flow generator and the first water outlet portion may have a same size.

The flow generator may be located closest to the water inlet portion at a side where the internal channel is in contact with the chamber.

The turbidity sensor module may further include an internal inlet portion through which water discharged from the first water outlet portion flows into the chamber, wherein a size of the internal inlet portion may be greater than a size of the first water outlet portion.

The internal inlet portion may be formed on a line extending in a longitudinal direction of the internal channel.

A size of the first water outlet portion may be smaller than a size of the water inlet portion, and a size of the second water outlet portion may be greater than a size of the water inlet portion.

The water inlet portion and the second water outlet portion may have a same size, and the first water outlet portion may be smaller than a half of a length of the internal channel.

The light path may be formed parallel to the longitudinal direction of the internal channel.

The chamber may include a flat portion on which the light source unit or the light receiver is disposed, and an inclined portion disposed between the flat portion and the second water outlet portion.

The water dispensing apparatus may further include: a water outlet through which the purified water is discharged; a water discharge passage guiding the purified water to the water outlet; and a drain passage which is branched from the water discharge passage between the water quality measurement unit and the water outlet, and through which the raw water or the purified water is drained.

The water dispensing apparatus may further include a water discharge valve selectively supplying the raw water or the purified water to the water discharge passage and the drain passage.

The water dispensing apparatus may further include: a hot water passage having one side branched from the purified water passage; a hot water module provided in the hot water passage and configured to heat purified water passing through the hot water passage; a cold water passage having one side branched from the purified water passage; and a cold water module provided in the cold water passage and configured to cool purified water passing through the cold water passage.

The water dispensing apparatus may further include a sensing passage which is branched from the water supply passage, and through which the raw water flows, wherein the water quality measurement unit is configured to measure water quality of the purified water in response to the purified water being introduced through the purified water passage, and configured to measure water quality of the raw water in response to the raw water being introduced through the sensing passage.

Upon measuring the water quality of the raw water, the water quality measurement unit may be configured to measure the water quality of the purified water after performing a rinse operation in which the purified water passes through the water quality measurement unit one or more times.

The water dispensing apparatus may further include: a switching valve configured to supply the raw water to the water supply passage or the sterilization passage; and a sensing valve configured to open and close the sensing passage.

### Advantageous Effects of Disclosure

According to at least one of the embodiments of the present disclosure, water quality may be measured more accurately.

According to at least one of the embodiments of the present disclosure, water quality of raw water and purified water may be measured more accurately.

According to at least one of the embodiments of the present disclosure, bubbles may be effectively separated and discharged.

According to at least one of the embodiments of the present disclosure, a passage may be managed hygienically by automatically detecting abnormal water quality and performing sterilization.

According to at least one of the embodiments of the present disclosure, sensing accuracy and efficiency may be improved by a rinse operation and water pipe configuration for sharing a sensor.

Meanwhile, various other effects will be directly or implicitly disclosed in the following detailed description of embodiments of the present disclosure.

### BRIEF DESCRIPTION OF DRAWINGS

FIG. 1 is a block diagram of main components of a water dispensing apparatus according to an embodiment of the present disclosure.

FIG. 2 is a conceptual diagram illustrating a water dispensing apparatus according to an embodiment of the present disclosure.

FIGS. 3 to 5 are diagrams referred to in the description of operation of the water dispensing apparatus of FIG. 2.

FIG. 6 is a conceptual diagram illustrating a water dispensing apparatus according to an embodiment of the present disclosure.

FIG. 7 is a diagram referred to in the description of optical turbidity sensing using light.

FIGS. 8 to 12 are diagrams referred to in the description of a turbidity sensor module according to various embodiments of the present disclosure.

### MODE OF DISCLOSURE

Hereinafter, embodiments of the present disclosure will be described in detail with reference to the accompanying drawings. However, it is understood that the present disclosure is not limited to these embodiments and may be modified in various forms.

In the drawings, in order to clearly and briefly describe embodiments of the present disclosure, the illustration of parts irrelevant to the description is omitted, and the same reference numerals are used for the same or extremely similar parts throughout the specification.

Hereinafter, the suffixes “module” and “unit” of elements herein are used for convenience of description and thus may be used interchangeably and do not have any distinguishable meanings or functions. Thus, the terms “module” and “unit” may be interchangeably used.

It will be understood that, although the terms “first”, “second”, etc. may be used herein to describe various elements, these elements should not be limited by these terms. These terms are only used to distinguish one element from another element.

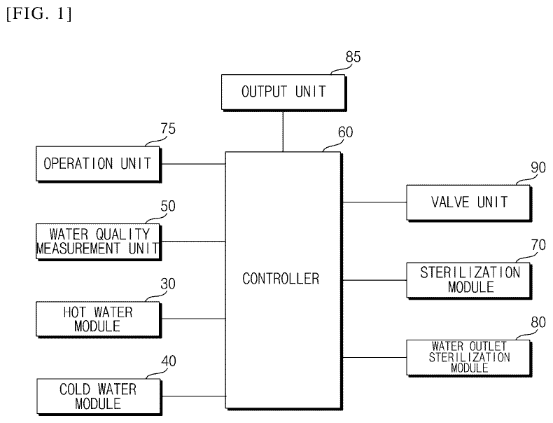

FIG. 1 is a block diagram of main components of a water dispensing apparatus according to an embodiment of the present disclosure.

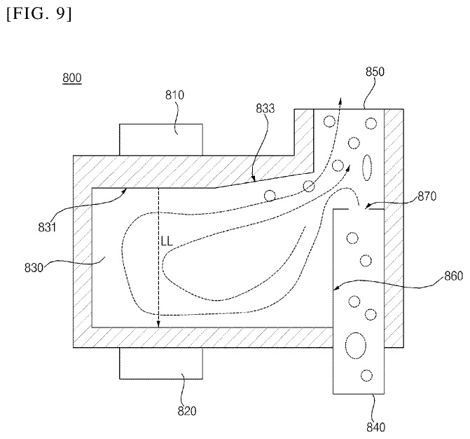

Referring to FIG. 1, the water dispensing apparatus according to an embodiment of the present disclosure includes a water quality measurement unit 50. The water quality measurement unit 50 may include a turbidity sensor module 55 (see FIG. 9). According to an embodiment of the present disclosure, turbidity (contamination level) may be detected in an optical sensing manner. A transmitted light method and a scattered light method are used as an optical sensing method for measuring turbidity. The transmitted light method senses turbidity by emitting light to a fluid, receiving light having passed through the fluid, and processing data. The scattered light method senses turbidity by receiving scattered light and converting it into data, and the scattered light method is classified according to a method of generating scattered light and a method of processing the received light data. The turbidity sensor module 800 according to an embodiment of the present disclosure may emit light to some of raw water or purified water, and may sense turbidity based on a received scattered light pattern. According to an embodiment of the present disclosure, particles and microorganisms are separated by patterning the movement and intensity of light scattered by microorganisms, and big data processing is performed to distinguish between types of indicator microorganisms according to water quality/sanitation standards. In addition, detected microbial concentration values and safety indicators are provided on a display so as to be intuitively identified by a user.

In addition, the water quality measurement unit 50 may employ a water quality measurement sensor, such as a turbidity sensor, a microbial sensor, a TDS sensor, etc., in order to detect contamination of water in a passage. The water quality measurement unit 50 may include at least one of a turbidity sensor, a microbial detection sensor, a chlorine sensor, a Total Dissolved Solids (TDS) sensor, and a Biochemical Oxygen Demand (BOD) sensor, and may measure at least one of the turbidity, microorganisms, residual chlorine, Total Dissolved Solids (TDS), and dissolved oxygen of introduced water. At least one of the sensors included in the water quality measurement unit 50 may be a shared sensor that measures the water quality of both raw water and purified water.

In the present disclosure, the flow of purified water or raw water into the water quality measurement unit 50 does not only mean that purified water or raw water flows into the water quality measurement unit 50. For example, some of the purified water or raw water may be sampled for measuring water quality in the water quality measurement unit 50, and then may be discharged after the measurement. In addition, at least some of the sensors included in the water quality measurement unit 50 may measure water quality of a liquid that flows. In this case, the flow of purified water or raw water into the water quality measurement unit 50 may mean that at least some of the purified water or raw water passes through a sensing capable region of the water quality measurement unit 50.

For example, in the case where the turbidity sensor module 800 includes an internal chamber, and when water flows into the internal chamber connected to a passage 20 and fills the chamber, the turbidity sensor module 800 may measure water quality by emitting light to the water that fills the chamber and receiving a scattered light pattern. In addition, the water inside the internal chamber may be discharged after measuring the water quality. Alternatively, a light source and a light receiver of the turbidity sensor module 800 may be positioned in a specific passage section (e.g., a section after a purified water passage 20 and a sensing passage 12 are joined), to emit light to the purified water or raw water that passes through the specific passage section and to receive a scattered light pattern.

The water dispensing apparatus according to an embodiment of the present disclosure may measure water quality of raw water and purified water by using a shared sensor for the same measurement items, such as turbidity and the like. The turbidity sensor module may measure the turbidity of raw water and the turbidity of purified water and transmit the sensing data to a controller 60. The controller 60 may control other components of the water dispensing apparatus based on the sensing data of the water quality measurement unit 50, such as a turbidity sensor and the like.

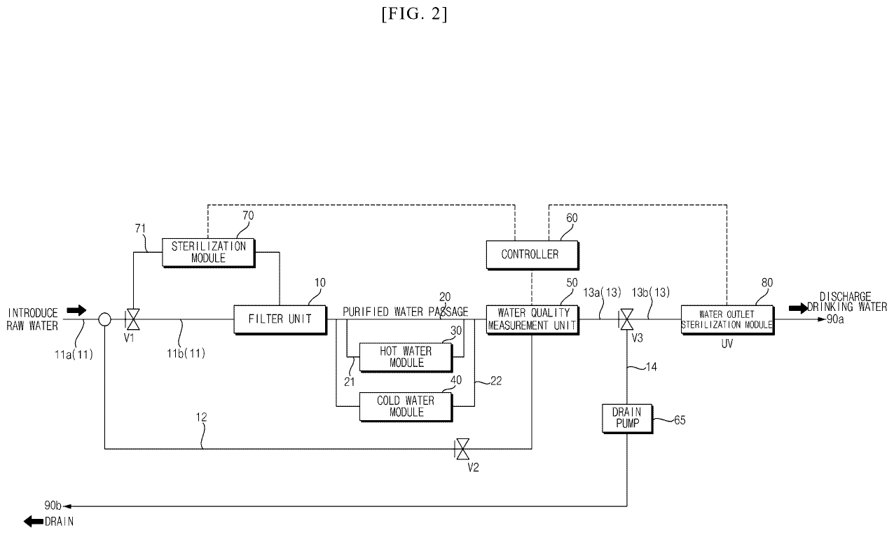

The water dispensing apparatus includes a filter 10 (see FIG. 2, etc.) configured to generate purified water by filtering raw water supplied from a water source. The filter 10 is provided to purify the supplied raw water and filter out various impurities and harmful substances contained in the raw water. One or more filters 10 are provided, and in the case where a plurality of filters are provided, a combination of filters having various functions may be used. For example, three filters 10 may be provided, including a pre-carbon filter, post-carbon filter, and a membrane filter or a hollow fiber membrane filter that is disposed between the pre-carbon filter and post-carbon filter. Alternatively, the filter 10 may include a pre-carbon filter and a UF composite filter.

Purified water purified by the filter 10 flows to a storage tank or a passage 20 (see FIG. 2, etc.). As water stored in the storage tank provides an environment suitable for propagation of microorganisms over time, it is more preferable that the water immediately flows through the passage 20. The purified water having passed through the filter 10 flows to the purified water passage 20.

In addition, the water dispensing apparatus includes a valve unit 90 including valves for controlling the flow of water. The valve unit 90 may include a plurality of valves V1, V2, V3, etc., which will be described later.

When the water quality measurement unit 50 measures the water quality of the raw water, the controller 60 may control the valve unit 90 and the like so that a rinse operation is performed in which the purified water passes through the water quality measurement unit 50 one or more times.

The water quality measurement unit 50 measures the water quality of the purified water after performing the rinse operation, thereby minimizing the effect of raw water on the water quality measurement of the purified water. Accordingly, by using only one water quality sensor of the same type, it is possible to efficiently and accurately sense each of the raw water and the purified water.

When tap water and purified water are discharged, they normally have low water contamination levels, and in order to measure low contamination concentrations, it is important to minimize the deviation of measurements. According to an embodiment of the present disclosure, by providing and controlling passages capable of simultaneously measuring the raw water and discharged water using one sensor, the increase in material costs may be minimized and the product may be manufactured in a compact size, compared to techniques that separately measure raw water and discharged water.

The water quality of tap water (raw water) and water quality of a water purifier (purified water) mostly fall into a low concentration range, and in order to clearly show a performance difference between the raw water and discharge water, it is most important to minimize the measurement deviation between devices by comparing using one sensor.

In addition, the water dispensing apparatus may include a hot water module 30 and a cold water module 40 configured to supply hot water/cold water. The hot water module 30 heats purified water, and then discharges the water toward a water outlet 90a (see FIG. 2, etc.). The cold water module 40 cools the purified water, and then discharges the water toward the water outlet 90a.

In addition, the water dispensing apparatus further includes an operation unit 75 and an output unit 85.

The operation unit 75 may receive user input and include one or more buttons. For example, the operation unit 75 may be provided as a touch panel and may include a capacity button for selecting a capacity of water to be discharged, a hot water button for selecting hot water and temperature of the hot water to be discharged, a purified water button for selecting purified water, a cold water button for selecting cold water, and other function buttons.

The output unit 85 may include a display device, such as a display (not shown) or a light emitting diode (LED) (not shown), and the like. For example, the output unit 85 may display information such as the operating state of the water dispensing apparatus, operating state related to error occurrence or the like, or water contamination level, etc.

The output unit 85 may include an audio device, such as a speaker (not shown), a buzzer (not shown), and the like. For example, the output unit 85 may output a sound effect for the operating state of the water dispensing apparatus and output a predetermined warning sound when an error occurs.

In addition, the water dispensing apparatus may further include modules for sanitation. For example, the water dispensing apparatus includes a sterilization module 70 using high-temperature or hot water. In addition, the water dispensing apparatus includes a water outlet sterilization module 80 for sterilization on the side of the water outlet 90a where there is a high possibility of contamination.

The sterilization module 70 instantly heats water to a high temperature to sterilize bacteria growing in water. In addition, the controller 60 may also operate the sterilization module 70 so that sterilized water (hot water) discharged from the sterilization module 70 may circulate through another passage to sterilize the passage. Based on water quality data measured by the water quality measurement unit 50, the controller 60 may control the hot water, discharged from the sterilization module 70, to flow to different passage regions to perform a sterilization operation for each passage region.

The water outlet sterilization module 80 may remove bacteria or viruses by emitting ultraviolet (UV) rays toward the water outlet 90a. The water outlet sterilization module 80 may include at least one Ultraviolet rays (UV) lamp or at least one Ultraviolet rays Light Emitting Diode (UV LED).

The water outlet sterilization module 80 may be periodically driven under control of the controller 60. Alternatively, the water outlet sterilization module 80 may be driven during a predetermined period of time before water is discharged. More preferably, based on water quality data measured by the water quality measurement unit 50, the controller 60 may drive the water output sterilization module 80 only when it is required, thereby improving efficiency. For example, the controller 60 may control the water outlet sterilization module 80 based on a result of purified water quality measurement.

The controller 60 may be connected to each component provided in the water dispensing apparatus. For example, the controller 60 may transmit and/or receive signals with the respective components provided in the water dispensing apparatus and may control the overall operation of the respective components.

The controller 60 may include at least one processor, and may control the overall operation of the water dispensing apparatus by using the processor included therein. Here, the processor may be a general processor such as a central processing unit (CPU). Obviously, the processor may be a dedicated device such as an ASIC or another hardware-based processor.

The controller 60 may perform various operations based on data received from the water quality measurement unit 50 including various sensors such as the turbidity sensor 800 and the like. In addition, the controller 60 may store data received from the water quality measurement unit 50 in a memory (not shown).

The water quality measurement unit 50 may measure water quality and output it to the controller 60. The controller 60 may perform a feedback operation in response to the received raw water and/or purified water quality measurement data. Alternatively, the water quality measurement unit 50 may directly determine a contamination level and transmit it to the controller 60, and the controller 60 may control other components to perform appropriate feedback operations based on the received contamination level.

By identifying the contamination state of raw water and/or purified water, the controller 60 may control the output unit 85 to provide a user with cleaning alarm or information about filter replacement period.

In addition, the controller 60 may detect odor that may occur based on a contamination level of raw water and/or purified water, and may perform an automatic cleaning/sterilization logic by operating the sterilization module 70 and the water outlet sterilization module 80 before a customer recognizes it. Accordingly, it is possible to improve user convenience and hygiene for non-professional users.

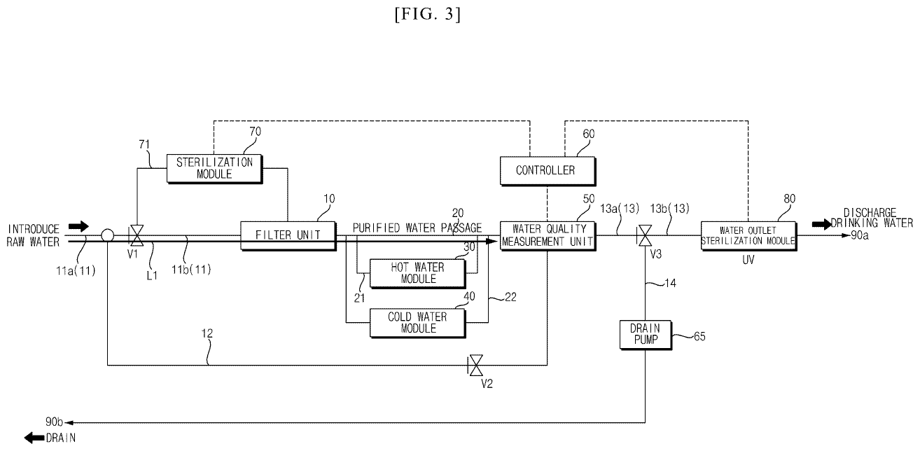

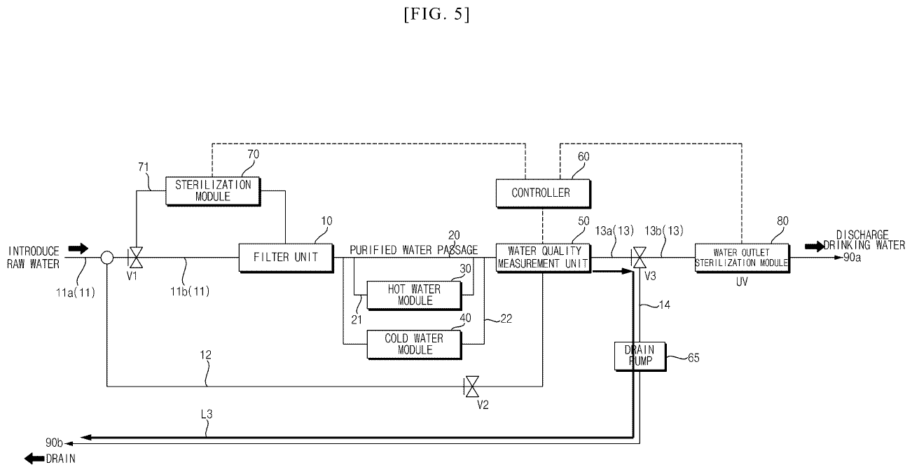

FIG. 2 is a conceptual diagram illustrating a water dispensing apparatus according to an embodiment of the present disclosure, and FIGS. 3 to 5 are diagrams referred to in the description of operation of the water dispensing apparatus of FIG. 2.

Referring to FIG. 2, the water dispensing apparatus includes a water supply passage 11 through which raw water supplied from a water source flows, and a filter unit 10 configured to generate purified water by filtering the raw water supplied through the water supply passage 11.

The purified water having passed through the filter unit 10 may flow toward the water outlet 90a through the purified water passage 20. The purified water having passed through the filter unit 10 may flow into the water quality measurement unit 50. When the purified water is introduced, the water quality measurement unit 50 may measure water quality of the purified water.

In addition, a sensing passage 12 may be branched from the water supply passage 11 such that the raw water may directly flow into the water quality measurement unit 50 through the sensing passage 12. When the raw water is introduced, the water quality measurement unit 50 may measure water quality of the raw water.

According to an embodiment of the present disclosure, a water supply valve V1 configured to control the supply of water toward the filter unit 10 and the purified water passage 20 may be disposed in the water supply passage 11. The water supply valve V1 may open and close the purified water passage 20. When the water supply valve V1 opens, the raw water is purified by passing through the filter unit 10 along a first line L1 of FIG. 3, and the purified water may flow into the water quality measurement unit 50 through the purified water passage 20.

Meanwhile, the water supply passage 11 may include a first water supply passage 11a connecting the water source and the water supply valve V1, and a second water supply passage 11b connecting the water supply valve V1 and the filter 10.

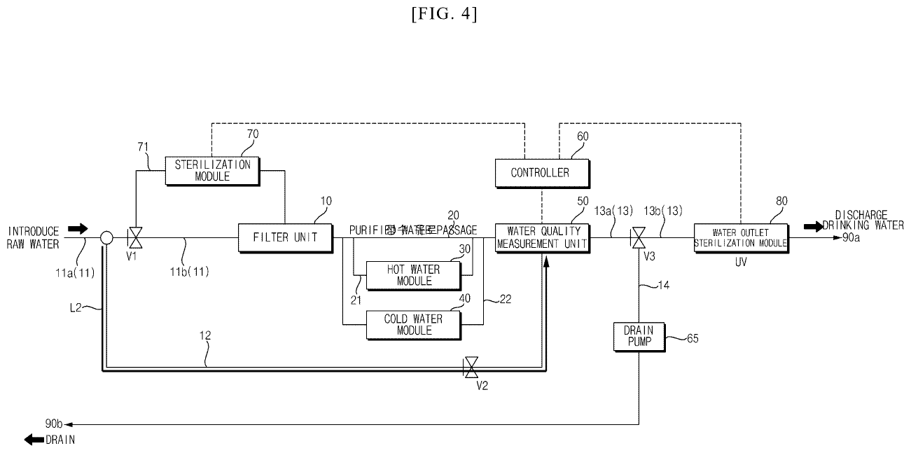

In addition, the sensing passage 12 may have a first end connected to the first water supply passage 11a, and a second end connected to the water quality measurement unit 50. A valve V2 configured to open and close the sensing passage 12 may be disposed in the sensing passage 12. When a sensing valve V2 opens, the raw water may directly flow into the water quality measurement unit 50 through the sensing passage 12 along a second line L2 of FIG. 4.

Referring to FIG. 2, the water dispensing apparatus may further include a water outlet 90a through which the purified water is discharged, a water discharge passage 13 guiding the purified water to the water outlet 90a, a drain passage 14 which is branched from the water discharge passage 13 between the water quality measurement unit 50 and the water outlet 90a and through which the raw water or the purified water is drained, and a water discharge valve V3 selectively supplying the raw water or the purified water to the water discharge passage 13 and the drain passage 14.

The water discharge valve V3 may divert water, for which water quality measurement is completed, to a drain 90b and the water outlet 90a under control of the controller 60. When a drain operation is performed, the water, for which the water quality measurement is completed, flows to the drain passage 14. By draining and cleaning the raw water, it is possible to prevent the raw water from being discharged as drinking water.

Meanwhile, the water discharge passage 13 may include a first water discharge passage 13a connecting the water quality measurement unit 50 and the water discharge valve V3, and a second water discharge passage 13b connecting the water discharge valve V3 and the water outlet 90a.

Meanwhile, the water outlet sterilization module 80 configured to emit UV rays to the water outlet 90a is disposed on the side of the water outlet 90a through which the purified water is discharged. The water outlet sterilization module 80 may sterilize the space of the water outlet and residual water. The controller 60 may operate the water outlet sterilization module 80 for a predetermined period of time based on the water quality data measured by the water quality measurement unit 50.

Referring to FIG. 2, a sterilization passage 71 may have a first side branched from the water supply passage 11 and a second side connected to the side of the filter 10, and the sterilization module 70 configured to heat water passing through the sterilization passage 71 is disposed in the sterilization passage 71. The controller 60 may operate the sterilization module 70 for a predetermined period of time based on the water quality data measured by the water quality measurement unit 50.

According to an embodiment of the present disclosure, the water supply valve V1 may be a switching valve selectively supplying the raw water to the water supply passage 11 or the sterilization passage 71.

Referring to FIG. 2, the water dispensing apparatus may further include a hot water passage 21 having one side branched from the purified water passage 20, a hot water module 30 provided in the hot water passage 21 and configured to heat purified water passing through the hot water passage 21, a cold water passage 22 having one side branched from the purified water passage 20, a cold water module 40 provided in the cold water passage 22 and configured to cool purified water passing through the cold water passage 22.

Referring to FIG. 2, the hot water passage 21 and the cold water passage 22 may join again the purified water passage 2. Alternatively, the hot water passage 21 and the cold water passage 22 may join again the water supply passage 13.

According to an embodiment of the present disclosure, a drain pump 65 may be disposed in the drain passage 14. By operating the drain pump 65 after the water quality is measured, the water, for which water quality measurement is performed, may be drained to the outside more rapidly at a faster rate.

In addition, the drain pump 65 may operate during a sterilization operation performed for each passage section. Accordingly, high-temperature or hot water after sterilization may be discharged more rapidly to the outside. Particularly, when the water discharge passage 13 connected to a cork on the side of the water outlet 90a is sterilized, some of the hot water is discharged toward the water outlet 90a, but a large amount of hot water may be discharged toward the drain 90b. Accordingly, it is possible to prevent safety accidents that may occur when a large amount of hot water is discharged, user discomfort, and inconvenience of a user having to deal with a large amount of hot water.

Meanwhile, when the purified water is introduced through the purified water passage 20, the water quality measurement unit 50 may measure water quality of the purified water, and when the raw water is introduced through the sensing passage 12, the water quality measurement unit 50 may measure water quality of the raw water.

Upon measuring the water quality of the raw water, the water quality measurement unit 50 may measure the water quality of the purified water after performing a rinse operation in which the purified water passes through the water quality measurement unit 50 one or more times. As described above, sensing accuracy and efficiency can be improved by a rinse operation and water pipe configuration for sharing a sensor.

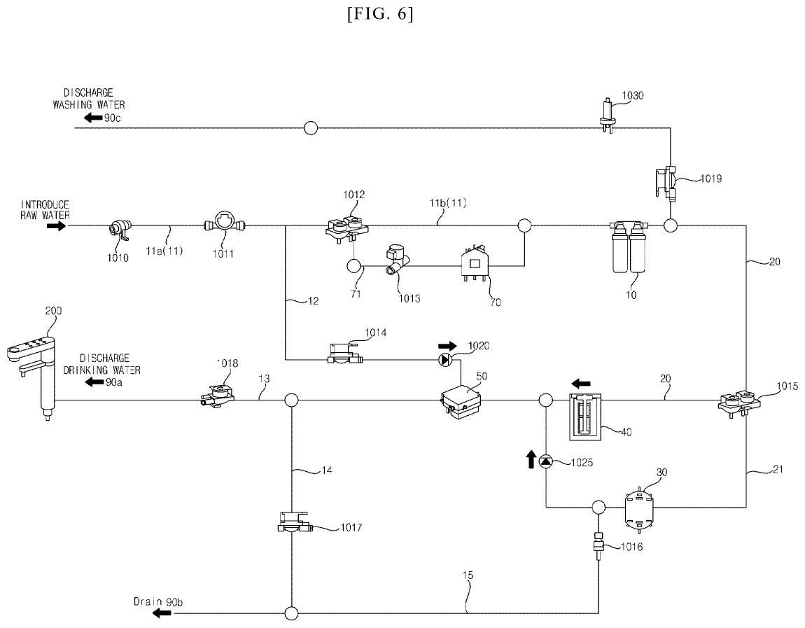

FIG. 6 is a conceptual diagram illustrating a water dispensing apparatus according to an embodiment of the present disclosure.

The water dispensing apparatus according to an embodiment of the present disclosure may correspond to various water treatment devices and purification devices, such as purifiers, refrigerators, etc., in which water is introduced from an external source and the introduced water is purified, and then is discharged.

For example, the water dispensing apparatus may be provided as an under sink type water purifier, with at least a portion thereof being disposed in a space under a kitchen sink.

Referring to FIG. 6, the water dispensing apparatus according to an embodiment of the present disclosure may include a water discharge part 200 having at least a portion exposed to the outside of the sink, and a remaining body part installed inside the sink.

The water dispensing apparatus includes a water supply passage 11 guiding raw water supplied from the outside to the inside, a filter 10 configured to purify the raw water supplied along the water supply passage 11 into purified water, and a purified water passage 20 through which purified water, having passed through the filter 10, flows toward the water discharge part 200.

Meanwhile, the water supply passage 11 connects an external water source and the filter 10. Raw water, supplied from an external water source, may be supplied to the filter 10 through the water supply passage 11.

The water (raw water) supplied to the filter 10 may pass through the filter 10 to be purified into purified water. At least one filter 10 may be provided. For example, a plurality of filters 10 may be provided. Accordingly, water having passed through the water supply passage 11 may be purified into cleaner water by passing through the plurality of filters 10.

In addition, the purified water having passed through the filter 10 may flow toward the water discharge part 200, which is exposed to the outside of the sink 10, through the purified water passage 20.

To this end, a first end of the purified water passage 20 may be connected to the filter 10, and a second end thereof may be connected to the water discharge part 200. Meanwhile, at least one of the cold water passage 22, the hot water passage 21, and a washing water passage 90c may be branched from the purified water passage 20.

In FIG. 6, an example is illustrated in which the cold water passage 22 is integrated into the purified water passage 20, and the hot water passage 21 and the washing water passage 90c are branched from the purified water passage 20.

One end of the purified water passage 20 is connected to the filter 10, and water having passed through the filter 10 flows toward the water discharge part 200 through the connected water discharge passage 13. The water discharge part 200 includes the water outlet 90a, and may dispense purified water and the like.

Water may pass through a washing water module 1030 provided in the washing water passage 90c, to be supplied in the state of sterilized water toward a washing water outlet. In the case where the water discharge part 200 includes a plurality of water outlets, the washing water outlet may also be formed in the water discharge part 200 in some examples.

Meanwhile, a decompression valve 1010 for adjusting a flow rate of water supplied to the filter 10 may be installed in the water supply passage 11.

In addition, at least one of a flow rate sensor 1011 for detecting a flow rate of water, a feed valve 35 for adjusting a flow rate of water or regulating the flow of water, or a flow speed sensor (not shown) for detecting a flow speed of the water may be installed in the water supply passage 11 or the purified water passage 20.

In addition, an opening/closing valve for regulating a flow of water in each passage may be installed separately in the purified water passage 20, the hot water passage 21, and the washing water passage 90c. For example, a washing water valve 1019 may be disposed in the washing water passage 90c.

Alternatively, a cold/hot/purified water valve 1015 for selectively supplying purified water to the purified water passage 20 and the hot water passage 21 may be installed at a branch point of the purified water passage 20 and the hot water passage 21. In addition,

In addition, a safety device 1025, such as a device for preventing backflow and the like, may be installed in the hot water passage 21. In addition, a safety valve 1016 that discharges steam may be installed in the hot water module 30. The steam of the hot water module 30 may be drained toward the drain 90b through a connected passage 15.

Meanwhile, a water discharge valve 1018 is disposed in the water discharge passage 13 to supply purified water, cold water, and hot water, which flow toward the water discharge part 200, to the water discharge part 200 or may block the water.

In addition, the drain passage 14 may be branched from the water discharge passage 13, or a drain valve 1017 may be disposed in the drain passage 14 to discharge purified water, cold water, hot water, and raw water toward the drain 90b.

Meanwhile, for example, each of the water discharge valve 1018 and the drain valve 1017 may be implemented with a 3-way valve that has one inlet, a first outlet and a second outlet that are selectively opened and includes an actuator that selectively opens and closes the two outlets. In this case, the first outlet may be connected to the water discharge part 200, and the second outlet may be connected to the drain 90b.

Meanwhile, raw water is supplied through the water supply passage 11 which is connected to a water source such as a water pipe, a water tank, or an underground pipe. A decompression valve 1010 is installed in the water supply passage 11, and the raw water passes through the decompression valve 1010 such that the pressure is reduced to a set pressure.

Further, the raw water, from which foreign substances are removed while passing through the filter 10, is converted into purified water. The purified water flows along the purified water passage 20. Further, the water may be branched into cold water-purified water and hot water.

First, the purified water branched into the cold water-purified water is branched again into cold water and purified water, and purified water or cold water may be supplied to a user through the water discharge part 200 based on the operation of the cold water module 40 by a user's operation of selecting purified water or cold water.

When a user requests discharge of cold water, the purified water passes through a cooling coil inside the cold water module 40. The water flowing along the cooling coil is heat-exchanged with a coolant within the cold water module 40 and then is cooled. To this end, the coolant is continuously cooled to maintain a set temperature. For reference, a compressor may be driven to cool the coolant. The driving of the compressor may be determined by a cold water temperature sensor provided in the cold water module 40. Thus, the coolant may be always maintained at the preset temperature, and to this end, the driving of the compressor may be controlled. The compressor may be adjusted in frequency to correspond to a load that is required for an inverter compressor and adjusted in cooling capacity. That is, the compressor may be driven by an inverter control to cool the coolant with optimal efficiency.

Meanwhile, when a user requests discharge of hot water, the water may be heated to a set temperature while passing through the hot water module 30. The hot water module 30 may be heated by an induction heating method, and to this end, an output of a working coil provided in the hot water module 30 may be adjusted. The purified water passing through the hot water module 30 may be heated at the set temperature. The hot water heated while passing through the hot water module 30 may flow toward the water discharge part 200.

Meanwhile, the sterilization passage 71 has a first side branched from the water supply passage 11, and a second side connected to the filter 10. A sterilization module 70 configured to heat water passing through the sterilization passage 71 and a flow control valve 1013 configured to adjust an amount of water flowing into the sterilization passage 71 may be disposed in the sterilization module 70.

Meanwhile, a feed valve 1012 for selectively supplying the raw water to the water supply passage 11 or the sterilization passage 71 may be disposed at a position where the sterilization passage 71 is branched from the water supply passage 11.

Meanwhile, the water supply passage 11 may include a first water supply passage 11a connecting the water source and the feed valve 1012, and a second water supply passage 11b connecting the feed valve 1012 and the filter 10.

Meanwhile, the sensing passage 12 described above may be branched from the first water supply passage 11a at a front end of the feed valve 1012. A sensing valve 1014 configured to open and close the sensing passage 12 and a backflow prevention device 1020 configured to prevent the backflow of raw water may be disposed in the sensing passage 12.

The controller 60 may close the feed valve 1012 and open the sensing valve 1014 to control the raw water to be supplied to the water quality measurement unit 50 through the sensor passage 14.

After measuring the water quality of the raw water, the controller 60 may close the water discharge valve 1018 and open the drain valve 1017 to discharge the raw water, which is measured by the water quality measurement unit 50, toward the drain 90b.

The controller 60 may open the feed valve 1012 toward the purified water passage 20 and close the sensing valve 1014 to control purified water to be supplied to the water quality measurement unit 50.

In addition, the controller 60 may close the water discharge valve 1018 and open the drain valve 1017 to perform a rinse operation by controlling the purified water, having passed through the water quality measurement unit 50, to be discharged toward the drain 90.

Then, the controller 60 may control the valves in the same manner to supply the purified water to the water quality measurement unit 50 for quality measurement of the purified water. Accordingly, it is possible to remove the effect of raw water, and the water quality of the purified water may be accurately measured by the same water quality measurement unit 50.

As described above, the water quality measurement unit 50 includes the turbidity sensor module. The turbidity sensor module may emit light to some of the raw water or the purified water, and may sense turbidity based on a received scattered light pattern. For example, the turbidity sensor module may detect the scattered light emanating from a visible light laser source and reflected and dispersed by floating substances in water, and output it as a signal value.

Scattered light increases in proportion to the amount of particles in a fluid, and may also be affected by external noise generated by the external environment such as particle behavior or fluid state (physical environment such as formation of bubbles or vortices), vibration, and the like. Regardless of the amount of particles, a signal value measured by the turbidity sensor may be exaggerated/reduced due to external noise that may affect a scattered light signal.

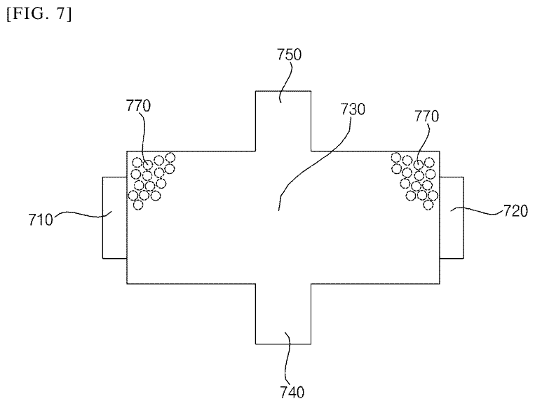

FIG. 7 is a diagram referred to in the description of optical turbidity sensing using light.

Referring to FIG. 7, water flows into a chamber 730 through an inlet 740, and the water inside the chamber 730 is discharged through an outlet 750. A light source unit 710 including a light source and a light receiver 720 configured to receive light are disposed to face each other on the left side or the right side of the chamber 730.

Meanwhile, when foreign substances contained in water are measured using an optical method, abnormal signals may be detected due to bubbles in the water. Bubbles 730 may be generated as water flows from the inlet 740 to the chamber 730, and at least some of the bubbles 730 may be present in a light path formed between the light source unit 710 and the light receiver 720 during turbidity sensing. Referring to FIG. 7, as the light source unit 710/light receiver 720 and outlet/inlet 750 and 740 are vertically arranged, bubbles may be present in the light path, thereby causing signal disturbance.

In the case of using the optical signal and the scattered light method, the bubbles 730 may affect the scattered light, and the light receiver 20 may receive an over signal. Defective sensor information may lead to malfunction of a device and result in unnecessary repair, causing discomfort to users and reducing their satisfaction with the product.

The present disclosure proposes a method of separating and removing bubbles and measuring floating foreign matter, rather than external noise such as bubbles in water and the like, thereby ensuring reliability of turbidity sensing.

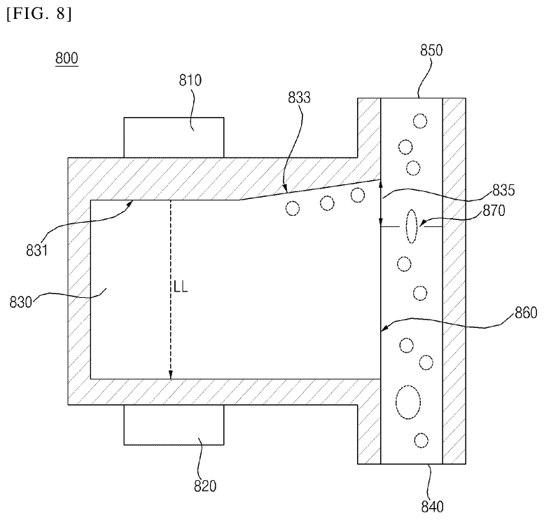

FIGS. 8 to 12 are diagrams referred to in the description of a turbidity sensor module according to various embodiments of the present disclosure.

FIGS. 8 to 12 illustrate the turbidity sensor module 800 included in the water quality measuring unit 50 described with reference to FIGS. 1 to 6.

Referring to FIGS. 8 to 12, the turbidity sensor module 800 includes a water inlet portion 840 for receiving water, an internal channel 860 through which water introduced through the water inlet portion 840 flows, a chamber 830 filled with water discharged from the internal channel 860, a light source unit 810 including a light source configured to emit light into the chamber 830, and a light receiver 820 configured to receive scattered light that is scattered from the chamber 830.

In the existing optical turbidity sensing, turbidity is determined by measuring an absolute variation in scattered light that forms 90 degrees with respect to incident light. In the most commercialized method, the intensity of scattered light is measured once per pixel, in which accessories (lenses, light source temperature control device, reflective structure, etc.) that maintain light output are required such that a precise measuring instrument is expensive and large. In addition, it is greatly affected by noise (biofilm, scale, etc.) that affects light source intensity.

The turbidity sensor module 800 may sense turbidity based on a pattern of the received scattered light. For example, the turbidity sensor module 800 may determine turbidity by measuring a relative change in a speckle image acquired by continuously capturing, with a camera module of the light receiver 820, an image of light scattered from the incident light emitted by the light source unit 810. By considering the relative change in the speckle image of the scattered light over time, it is not affected by stationary noise (scale, etc.), and no additional optical accessories are required, thereby allowing for a compact size and low price.

However, when foreign substances (turbidity) in water are measured, bubbles formed when a fluid is introduced may cause an unnecessary optical signal, such that a measured turbidity may be exaggerated. The bubbles contained in water may affect an optical signal of the turbidity sensor, such that a signal is detected higher than an actual turbidity value, thereby reducing sensing accuracy, and the turbidity sensor may transmit defective information to the water dispensing apparatus and an actual user.

In the present disclosure, a light path LL of light emitted by the light source unit 810 is spaced apart from the internal channel 860, such that even when bubbles are formed by water introduced through the water inlet portion 810, the bubbles may be separated from the light path LL as much as possible.

More specifically, the internal channel 860 primarily filters out bubbles generated by the introduced water, and may reduce the amount of the bubbles flowing into the chamber 830. In addition, the internal channel 860 may guide an inflow/movement path of water so that the bubbles may not affect the light path LL. The bubbles may move toward the first and second water outlet portions 870 and 850 by inertia of motion.

There is a high probability of bubble formation when water flows through the water inlet portion 810. Accordingly, by connecting the internal channel 860 to the water inlet portion 840, and by controlling water, introduced through the water inlet portion 840, to flow into the chamber 830 through the internal channel 860, bubbles generated when a fluid flows into the internal chamber 830 of the turbidity sensor module 800 may be removed or separated from the light path LL. Accordingly, by eliminating the effect of bubbles, a good turbidity signal may be acquired, the occurrence of sensor abnormalities may be prevented in advance, and sensing accuracy/reliability may be improved.

Referring to FIGS. 8 to 12, the turbidity sensor module 800 may include a first water outlet portion 870 through which water is discharged from the internal channel 860 to the chamber 830, and a second water outlet portion 850 through which water is discharged from the chamber 830 to the outside. The internal channel 860 may be a conduit preventing bubbles, which may enter through the water inlet portion 840, and bubbles which may be generated during the inflow, from flowing into the chamber 830, and guiding water to the first and second water outlet portions 870 and 850.

The first water outlet portion 870 is a functional unit capable of adjusting the size of bubbles that may be present in water when water flowing through the water inlet portion 840 primarily passes through it. The first water outlet portion 870 may be greater than 0.5 times the diameter of a flow passage tube inside the water dispensing apparatus and less than 0.8 times the diameter. If the first water outlet portion 870 is too narrow, it impedes the flow of water, and thus should be greater than at least a half of the diameter of the flow passage tube, and if it is too large, it cannot separate bubbles, and thus is preferably less than 0.8 times the diameter of the flow passage tube.

The first water outlet portion 870 may be formed in an open portion of one surface of the internal channel 860 that faces the second water outlet portion 850. The internal channel 860 may have a long side formed in a direction in which water is introduced, and a short side formed in a direction perpendicular to the long side. The long side may be one surface of the internal channel 860 that faces the light path LL, and the short side may be one surface of the internal channel 860 that faces the second water outlet portion 850. The first water outlet portion 870 may be disposed at the short side of the internal channel 860.

Meanwhile, the internal channel 860 may be formed in the shape of a pipe so as to be easily connected to a water pipe of the water dispensing apparatus. In this case, the long side may be formed in a longitudinal direction of the internal channel 860.

The light source unit 810 may be disposed at an upper end of the chamber 830, and the light receiver 820 may be disposed at a lower end of the chamber 830. By contrast, the light source unit 810 may be disposed at a lower end of the chamber 830, and the light receiver 820 may be disposed at an upper end of the chamber 830. The light source unit 810 and the light receiver 820 are disposed at the upper and lower ends of the chamber 830 to form a light path LL in the up-down direction. In addition, the internal channel 860 may be spaced apart from the light source unit 810/light receiver 820 by a predetermined distance. Considering only the elimination of the effect of bubbles, it is preferable that internal channel 860 and the light source unit 810/light receiver 820 are spaced apart from each other on both sides of the chamber 830 by a maximum distance. In some examples, the light path LL may be formed parallel to the longitudinal direction of the internal channel 860, rather than perpendicular to the direction. In order to prevent bubbles from interfering with the light path LL, the light source unit 810/light receiver 820 and the inlet 840/outlet 850 may be aligned horizontally.

In a turbidity sensing zone Z1 including the light path LL, the light source unit 810 emits light and the light receiver 820 receives scattered light, and turbidity is sensed based on a scattered light pattern. Meanwhile, in order to minimize introduction of bubbles into the turbidity sensing zone Z1, the internal channel 860 is disposed at a front end of the turbidity sensing zone Z1.

Meanwhile, the water inlet portion 840 is connected to the flow passage tube inside the water dispensing apparatus, such that its size (diameter) a is preferably the same as the diameter of the flow passage tube inside the water dispensing apparatus.

Referring to FIG. 8, in the turbidity sensor module 800 according to an embodiment of the present disclosure, the water inlet portion 840 and the second water outlet portion 850 may have the same size (diameter). The shape and size of the water inlet portion 840 and the second water outlet portion 850 may correspond to the shape and size of the water pipe of the water dispensing apparatus.

Meanwhile, the chamber 830 may include a flat portion 831 on which the light source unit 810 or the light receiver 820 is disposed, and an inclined portion 833 disposed between the flat portion 831 and the second water outlet portion 850. The inclined portion 833 is inclined to guide bubbles that may escape from the first water outlet portion 870 to the second water outlet portion 850.

Referring to FIG. 9, in the turbidity sensor module 800 according to an embodiment of the present disclosure, a size of the first water outlet portion 870 may be smaller than a size of the water inlet portion 840, and a size of the second water outlet portion 850 may be greater than the size of the water inlet portion 840. In order to rapidly measure the turbidity of water flowing into the turbidity sensor module 800, it is required to ensure sufficient internal flow so that the sample is well mixed and flows smoothly toward the outlet (second water outlet portion 850). Accordingly, the size of the second water outlet portion 850 may be greater than the size of the water inlet portion 840. That is, an outlet diameter may be larger than an inlet diameter, thereby allowing water filled therein to be smoothly discharged through the outlet.

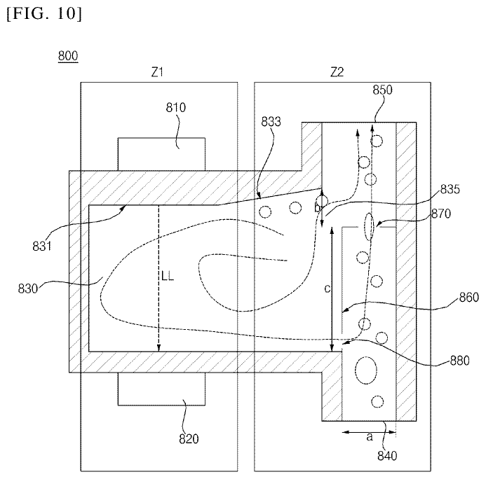

Referring to FIG. 10, the first water outlet portion 870 may be smaller than a half of a longitudinal length C of the internal channel 860. That is, the longitudinal length C of the internal channel 860 is twice as large as the size (diameter) of the first water outlet portion 870, thereby ensuring sufficient space inside the internal channel 860. In addition, the size of the second water outlet portion 850 may be smaller than a half of the length of the internal channel 830.

Referring to FIG. 10, the turbidity sensor module 800 according to an embodiment of the present disclosure may further include a flow generator 880 formed in an open portion of a side surface (long side) of the internal channel 860 that faces the light path LL. That is, two outlets 870 and 880 may be provided above/below a water inlet passage of the internal channel 860. Accordingly, water present therein flows into the flow generator 880 below the water inlet passage due to a pressure difference when water moves, such that internal flow occurs and water exchange may take place.

The flow generator 880 is a functional unit capable of separating bubbles that are present in water or can be generated therein when water flowing through the water inlet portion 840 passes through it. In some examples, the flow generator 880 and the first water outlet portion 870 may have the same size. That is, a size of the diameter of the flow generator 880 may be equal to a size of the first water outlet portion 870. The flow generator 880 may be located closest to the water inlet portion 840 at a side where the internal channel 860 is in contact with the chamber 830.

Referring to FIGS. 8 to 12, the turbidity sensor module 800 may further include an internal inlet portion 835 through which the water in the chamber 830 flows to the second water outlet portion 850.

The internal inlet portion 835 is a section in which water flowing from the internal channel 860 flows to the turbidity sensing zone 1 including the light path LL, and may also be a section in which the water in the chamber 830 flows to the second water outlet portion 850.

A size b of the internal inlet portion 835 may be greater than a size of the first water outlet portion 870. In order to allow water to smoothly flow into the turbidity sensing zone Z1, a height b of the internal inlet portion 835 may be preferably greater than or equal to at least 0.25*internal channel height c.

In some examples, the internal inlet portion 835 may be formed on a line extending in a longitudinal direction of the internal channel 860.

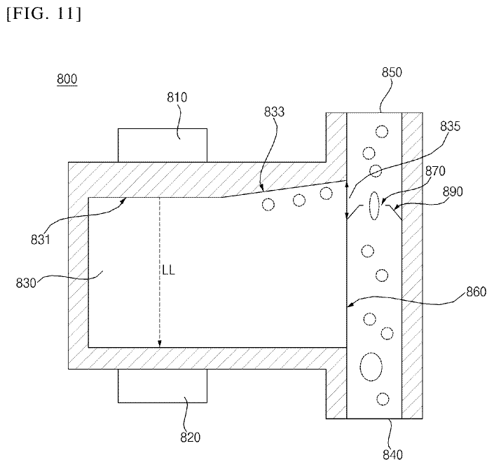

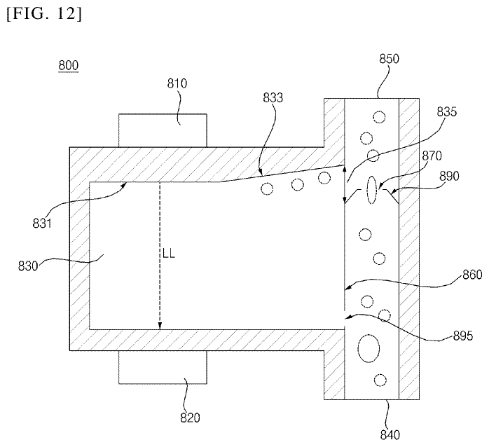

Referring to FIGS. 11 and 12, one surface of the internal channel 86 in which the first water outlet portion 870 is formed may include an inclined portion 890 inclined at both sides of the first water outlet portion 870. Accordingly, water may be discharged more smoothly through the first water outlet portion 870.

Even in this case, a flow generator 895 is further included, which is formed in an open portion of a side surface (long side) of the internal channel 860 that faces the light path LL. The flow generator 895 and the first water outlet portion 870 may have the same size. The flow generator 895 may be located closest to the water inlet portion 840 at a side where the internal channel 860 is in contact with the chamber 830.

It will be apparent that, although the preferred embodiments have been illustrated and described above, the present disclosure is not limited to the above-described specific embodiments, and various modifications and variations can be made by those skilled in the art without departing from the gist of the appended claims. Thus, it is intended that the modifications and variations should not be understood independently of the technical spirit or prospect of the present disclosure.

1. A water dispensing apparatus comprising:
a water supply passage through which raw water, supplied from a water source, flows;
a filter configured to generate purified water by filtering the raw water supplied through the water supply passage;
a purified water passage through which the purified water, having passed through the filter, flows; and
a water quality measurement unit connected to the purified water passage and configured to measure water quality of the purified water,
wherein the water quality measurement unit comprises a turbidity sensor module configured to measure turbidity of water introduced thereinto,
wherein the turbidity sensor module comprises:
a water inlet portion for receiving water;
an internal channel through which water, introduced through the water inlet portion, flows;
a chamber filled with water discharged from the internal channel;
a light source unit including a light source configured to emit light into the chamber; and
a light receiver configured to receive scattered light that is scattered from the chamber,
wherein a light path of light emitted by the light source unit is spaced apart from the internal channel.

2. The water dispensing apparatus of claim 1, wherein the turbidity sensor module comprises a first water outlet portion through which water is discharged from the internal channel to the chamber, and a second water outlet portion through which water is discharged from the chamber to an outside.

3. The water dispensing apparatus of claim 2, wherein the first water outlet portion is formed in an open portion of one surface of the internal channel that faces the second water outlet portion.

4. The water dispensing apparatus of claim 2, wherein one surface of the internal channel in which the first water outlet portion is formed has an inclined portion inclined at both sides of the first water outlet portion.

5. The water dispensing apparatus of claim 2, wherein a size of the second water outlet portion is smaller than a half of a length of the internal channel.

6. The water dispensing apparatus of claim 2, wherein the turbidity sensor module further comprises a flow generator formed in an open portion of a side surface of the internal channel that faces the light path.

7. The water dispensing apparatus of claim 6, wherein the flow generator and the first water outlet portion have a same size.

8. The water dispensing apparatus of claim 6, wherein the flow generator is located closest to the water inlet portion at a side where the internal channel is in contact with the chamber.

9. The water dispensing apparatus of claim 2, wherein the turbidity sensor module further comprises an internal inlet portion through which water discharged from the first water outlet portion flows into the chamber,
wherein a size of the internal inlet portion is greater than a size of the first water outlet portion.

10. The water dispensing apparatus of claim 9, wherein the internal inlet portion is formed on a line extending in a longitudinal direction of the internal channel.

11. The water dispensing apparatus of claim 2, wherein a size of the first water outlet portion is smaller than a size of the water inlet portion, and a size of the second water outlet portion is greater than a size of the water inlet portion.

12. The water dispensing apparatus of claim 2, wherein the water inlet portion and the second water outlet portion have a same size, and the first water outlet portion is smaller than a half of a length of the internal channel.

13. The water dispensing apparatus of claim 1, wherein the light path is formed parallel to the longitudinal direction of the internal channel.

14. The water dispensing apparatus of claim 1, wherein the chamber comprises a flat portion on which the light source unit or the light receiver is disposed, and an inclined portion disposed between the flat portion and the second water outlet portion.

15. The water dispensing apparatus of claim 1, further comprising:
a water outlet through which the purified water is discharged;
a water discharge passage guiding the purified water to the water outlet; and
a drain passage which is branched from the water discharge passage between the water quality measurement unit and the water outlet, and through which the raw water or the purified water is drained.

16. The water dispensing apparatus of claim 15, further comprising a water discharge valve selectively supplying the raw water or the purified water to the water discharge passage and the drain passage.

17. The water dispensing apparatus of claim 15, further comprising:
a hot water passage having one side branched from the purified water passage;
a hot water module provided in the hot water passage and configured to heat purified water passing through the hot water passage;
a cold water passage having one side branched from the purified water passage; and
a cold water module provided in the cold water passage and configured to cool purified water passing through the cold water passage.

18. The water dispensing apparatus of claim 1, further comprising a sensing passage which is branched from the water supply passage, and through which the raw water flows,
wherein the water quality measurement unit is configured to measure water quality of the purified water in response to the purified water being introduced through the purified water passage, and configured to measure water quality of the raw water in response to the raw water being introduced through the sensing passage.

19. The water dispensing apparatus of claim 18, wherein upon measuring the water quality of the raw water, the water quality measurement unit is configured to measure the water quality of the purified water after performing a rinse operation in which the purified water passes through the water quality measurement unit one or more times.

20. The water dispensing apparatus of claim 18, further comprising:
a switching valve configured to supply the raw water to the water supply passage or the sterilization passage; and
a sensing valve configured to open and close the sensing passage.
